(19) United States
(12) Patent Application Publication
(10) Pub. No.: US 2012/0093764 A1
(43) Pub. Date: Apr. 19, 2012

(54) TREATMENT OF DIABETES USING G-CSF AND HYPERBARIC OXYGEN

(76) Inventor: Dipnarine Maharaj, Boynton Beach, FL (US)

(21) Appl. No.: 12/907,567

(22) Filed: Oct. 19, 2010

(51) Int. Cl. A61K 38/19; A61P 9/08; A61P 3/10; A61P 3/00; A61K 33/00; A61K 35/12

(52) U.S. Cl. 424/85.1; 424/600; 424/93.7

(57) ABSTRACT

A method of treating endothelial cells comprising: inducing vasodilation in a patient; and administering a composition including a stem cell proliferation agent to the patient.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to a method for treating diabetes and other metabolic disorders.

### BACKGROUND OF THE INVENTION

Diabetes is a chronic metabolic disease affecting about 24 million people in the United

States and 200 million people worldwide. One of the most common and deadly conditions associated with diabetes is an increased risk of cardiovascular disease (CVD). In fact, current statistics indicate that about 75% of diabetic patients will die of CVD. Patients with Type I and Type II diabetes also have an increased risk for macrovascular and microvascular diseases, stroke, hypertension, and obesity. Current treatments for mitigating the progression and symptoms of diabetes include oral hypoglycemic drugs and insulin. Other potential treatments, such as Beta cell tissue transplant, are being investigated and have not yet been proven to be effective. And, while treatments for the symptoms of diabetes exist, there is currently no cure for the disease.

One significant cause of diabetes, hypertension, microvascluar and macrovascular, diseases, as well as obesity, is endothelial cell dysfunction. The endothelial cells line the entire circulatory system, from the heart to the smallest capillary. These cells reduce turbulent blood flood allowing the blood to be pumped farther. Owing to their importance to the vascular system, endothelial cell dysfunction is a major cause of CVD and stroke. In particular, dysfunctional endothelial cells increase the inflammatory response in the vascular system, which increases the progression of atherosclerosis and other cardiovascular maladies. However, the biochemical and cellular links between elevated blood glucose levels associated with diabetes and endothelial cell dysfunction remain incompletely understood. As such, the proliferation of functional endothelial cells has become a major target of diabetes and CVD researchers.

Current methods to increase the number of functional endothelial cells include increasing the number of stem cells produced in bone marrow. These stem cells can then be differentiated into fully functional endothelial cells. While preliminary results have indicated a positive effect of such treatments on blood glucose levels, current methods of increasing the number of stem cells have not been proven safe or effective.

Another avenue to stimulate endothelial cells includes locally increasing the production of nitric oxide (NO) by disposing a diabetic appendage into hyperbaric oxygen (HBO) to promote wound healing. The effectiveness of NO on a diabetic wound is twofold. First, the NO acts upon endothelial cells in the appendage to cause dilatation of blood vessels and inhibits vasoconstriction and subsequent hypertension. Second, NO has antimicrobial properties, which lowers the risk of infection. However, treatments of diabetic appendages using NO have only been applied to diabetic appendages. As such, their impact has been limited to treating damaged tissue regions and not the underlying disease.

Therefore, what is needed is treatment regimen for diabetes that is safe, effective, fast, and that treats diabetes and its symptoms throughout the entire body.

### SUMMARY OF THE INVENTION

The present invention advantageously provides a method for treating diabetes. The method includes inducing vasodilation in a patient; and administering a composition including a stem cell proliferation agent to the patient.

In another embodiment, the method includes administering hyperbaric oxygen to a patient for between five and ten consecutive days; administering a composition having G-CSF to the patient for about seven consecutive days after administering the hyperbaric oxygen; extracting stem cells from the patient after administering the hyperbaric oxygen and administering the composition; and infusing the extracted stem cells into the patient.

In yet another embodiment, the method includes measuring a blood glucose level of a patient with diabetes; determining one or more target levels for the patient's blood glucose level; administering hyperbaric oxygen to the patient for between five and ten consecutive days, the hyperbaric oxygen being inhaled by the patient; administering a composition having G-CSF at a dose of about 300 μg to about 960 μg per day to the patient for about seven consecutive days after administering the hyperbaric oxygen; extracting stem cells from the patient after the seven consecutive days of administering the composition; infusing the extracted stem cells into the patient; measuring the blood glucose levels; comparing the measured blood glucose levels to the one or more target blood glucose levels; and modifying at least one of the administering of the hyperbaric oxygen and the composition based on the comparison.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a flow chart illustrating an embodiment of a method of treating diabetes;

FIG. 2 is another embodiment of a method of treating diabetes and results of such a treatment on a patient with Type II diabetes;

FIG. 3 is another embodiment of a method of treating diabetes and results of such a treatment on a patient with Type I diabetes; and

FIG. 4 is another embodiment of a method of treating diabetes and results of such a treatment on a patient with Type II diabetes.

### DETAILED DESCRIPTION OF THE INVENTION

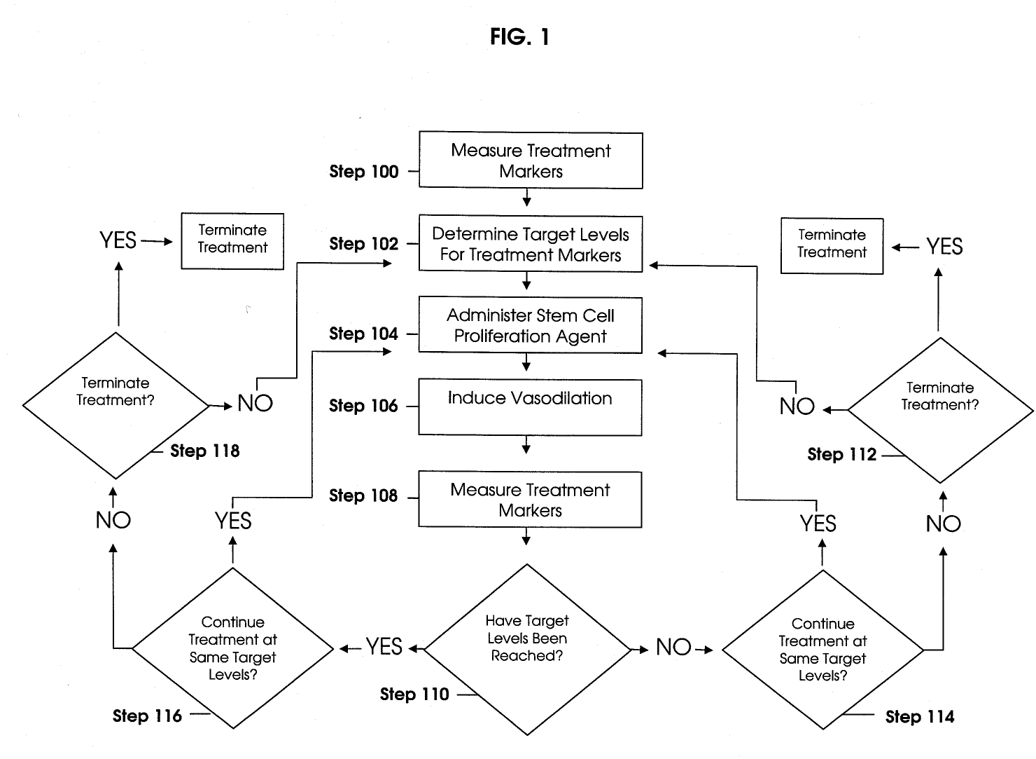

Now referring to the figures, where like reference designators refer to like steps, there is shown in FIG. 1 a method of treating the endothelial cells or Beta cells of a patient. The method may include measuring blood glucose levels, C-peptide levels, endothelial stem cell populations, and/or HbA1C levels in a patient's blood, among other diabetes or metabolic disorder indicators (collectively, “treatment markers”) before, during, and/or after the treatment methods described below (Step 100). For example, endothelial stem cell populations in the blood may be measured using flow cytometry in a human or animal patient. Urine analysis or genetic testing may also be performed to assess and determine the one or more treatment markers.

The method further includes determining a predetermined target level for the treatment markers, either based on the specific patient or based on universally accepted target treatment markers (Step 102). For example, a universal predetermined target level for blood glucose may be between 64.8 and 104.4 mg/dL. If the patient has particularly high normal blood glucose level compared to the average, the target level may be adjusted based on that individual. As such, the above treatment methods are dynamic in that they can be tailored for an individual patient.

The method further includes providing and/or administering a composition including granulocyte colony-stimulating factor (G-CSF), a derivative thereof, or any stem cell proliferation or stimulation agent, to a patient. (Step 104) They may patient have endothelial cell dysfunction, diabetes, or any metabolic disorder or cardiovascular disease, or alternatively may be healthy. The composition may be administered to the patient, for example, orally, by subcutaneous injection, by infusion into the blood, or delivered directly to a target tissue site. For example, a catheter may be used to transport and deliver a coated implant, for example, a stent with the composition to a blood vessel, or the composition may be surgically delivered to a target site, for example the pancreas or bone marrow, by implantation or graft. The composition may be delivered by a single dose, bolus, multiple injections, or by continuous infusion. For example, G-CSF may be injected, infused, or otherwise administered in the blood stream, bone marrow, pancreas, or any location in the body.

In an exemplary embodiment where G-CSF is administered, any formulation of G-CSF or other stem cell proliferation agents may be included in the composition and administered into the patient. Examples of other stem cell proliferation agents may include, for example, AMD 3100, CXCR4 antagonist, up regulator of metalloproteinase (MMP-9) expression, up regulator of VEGF, SDF-1, angiopoietin-1 over expression, granulocyte monocyte colony stimulating factor (GM-CSF), erythropoietin, 3-hydroxy-3-methylglutaryl coenzyme A reductase inhibitors, statins, peroxisome proliferator-activated receptor gamma agonists, placental growth factor, estrogen, VEGF-A, and/or VEGFR2. In an exemplary embodiment where G-CSF is administered, commercially available recombinant human G-CSF, for example, Neupogen™ may be used, Neulastam, recombinant G-CSF, or G-CSF produced from hamster ovary cells. A single source of G-CSF, or a combination of derivatives and sources of G-CSF, may be used in the composition. In an embodiment, the G-CSF administered is a glycoprotein with a molecular weight of 19.6 KDa. The G-CSF may be introduced into to the patient in any suitable form or formulation. For example, the G-CSF may be formulated in pharmaceutically acceptable carriers or diluents such as physiological saline or a buffered salt solution.

Supplements or other medications may be provided with the stem cell proliferation agent, for example, green tea, astragalus, goji berries, Lactobacillus fermentum, ellagic acid, beta 1,3 glucan, vitamin D3, carnosine, blueberries, arginine, may be provided in addition to the patient during or after the administration of the stem cell proliferation agent.

The method may further include inducing vasodilation in the patient, for example, by administering hyperbaric oxygen to the patient. (Step 106). It is further contemplated that the composition may be administered by any of the methods described below to a patient having any metabolic disorder, such as diabetes, and/or cardiovascular disease, or to a patient who exhibits the warning signs of these diseases, such as high blood pressure, high glucose levels, atherosclerosis, among other conditions.

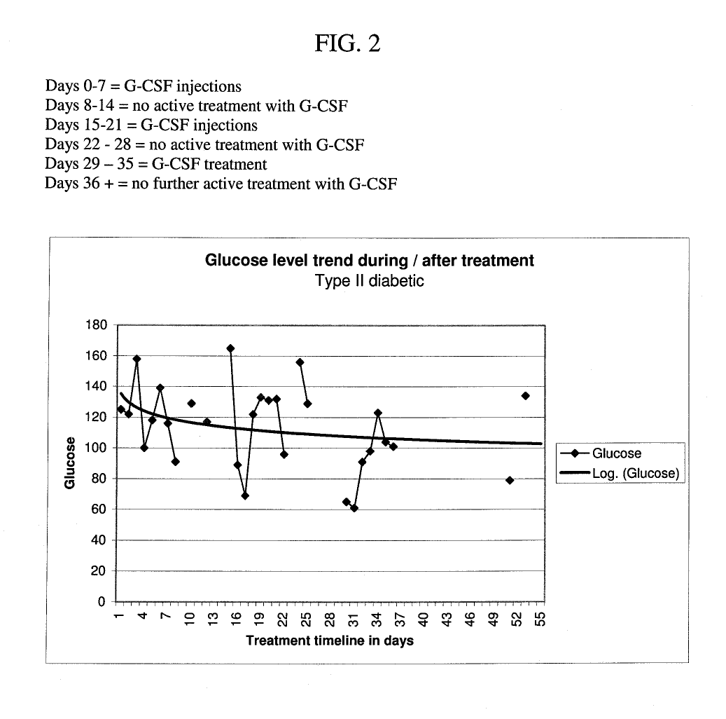

Referring now to FIG. 2, where an exemplary method of administering a composition having a stem cell proliferation agent to a patient with diabetes is shown. G-CSF, or a derivative or isomer thereof, may be subcutaneously injected, or otherwise administered into the blood stream or target tissue of the patient for a period of seven consecutive days. In this embodiment, the stem cells mobilize in response to the administered G-CSF and passively migrate into the blood stream. Optionally, after the seven day treatment period, stem cells may be extracted, for example, from the bone marrow or blood, and then infused intravenously into the blood stream or any location within the body within about 24 to 96 hours after the seven day stem cell proliferation agent treatment. The process of extracting the stem cells may be accomplished by, for example, apheresis. In an exemplary embodiment, about 2×10⁶ cells are extracted and returned to the blood stream, but any number or volume of stem cells may be extracted and returned to any part of the body, for example, the pancreas or blood stream. Alternatively, stem cells, for example, pluripotent stem cells may mobilize and migrate to the blood stream without extraction or reinfusion following administration of the stem cell proliferation agent. The administration of G-CSF thus may cause an increase in stem cell production and mobilization without the need to extract and reinfuse them into a particular organ or blood stream. In such an embodiment, pluripotent stem cells or fully mature cells may migrate to the pancreas via the blood stream, or any damaged organ.

Following the seven day stem cell proliferation agent treatment, the method includes a seven day period of rest, which when combined with the seven day stem cell proliferation agent treatment defines a cycle. During the rest period no new injections of stem cell proliferation agent treatment are administered, however, previously extracted stem cells may be returned intravenously to the patient for a few days. Optionally, additional injections of stem cell proliferation agent may be administered during the rest period. This treatment cycle may be repeated for a total of three cycles, but any number of cycles is contemplated. In this embodiment, after about the 42^{th} day (three cycles) no further treatments are administered. The number of treatments per day and the amount per dose may vary during each cycle. For example, depending on the formulation administered, the dose of G-CSF administered may range from about 300 μg to about 960 μg one a day, or from about 5 μg/kg to about 32 μg/kg once a day. The foregoing ranges are exemplary and may vary depending on the size, age, and health of the patient, the route of administration, the number and concentration of other medications the patient is taking, the severity of the patient's condition, the tolerance of the patient to the composition, among other factors. For example, a dose for 70 kg human may be 480 μg in a 2 ml injection may be an appropriate dose.

After each cycle, each day, or at the end of treatment, the stem cells may be extracted from the patient, typically from the bone marrow and or blood, and infused into the patient's blood stream over a period of time, for example, 24 to 96 hours. Alternatively, the stem cells can be extracted and frozen for reinfusion at a later date. Alternatively, the stem cells may be mobilized and passively migrate into the blood stream without extraction and reinfusion. As shown in FIG. 2, the results of a three cycle treatment show a marked decrease in blood glucoses levels after three cycles of treatment. The effect of the composition is also to increase functional endothelial and Beta cells.

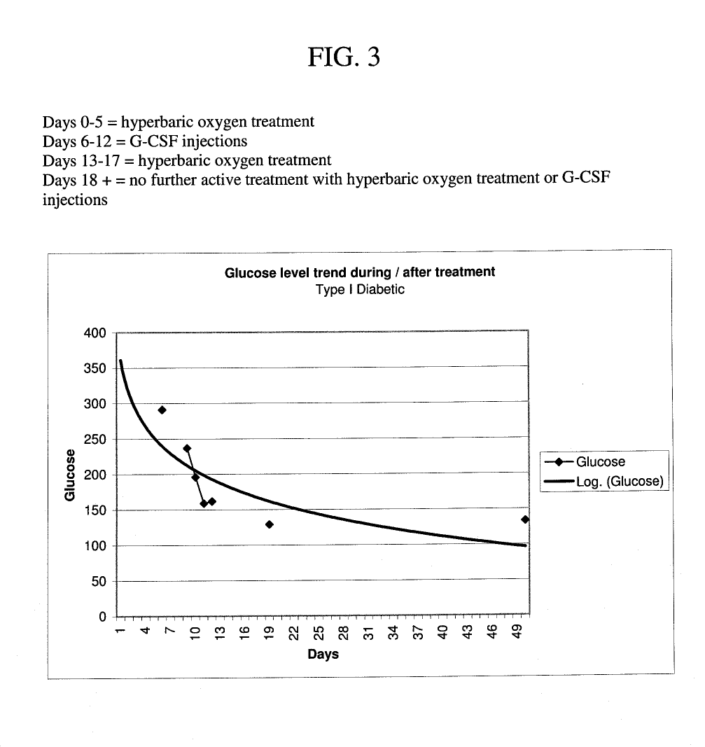

Referring now to FIG. 3, where another method of treating diabetes is shown. The method includes treating a patient with Type I diabetes with HBO treatment for a period of, for example, about five consecutive days, followed by treatments of a stem cell proliferation agent in accordance with the principles discussed above with respect to the method shown in FIG. 2. In an exemplary embodiment, a patient with Type I diabetes or any metabolic or cardiovascular disorder, is either fully or partially disposed within a hyperbaric chamber such that the patient may inhale high pressure oxygen or air. The hyperbaric chamber may be flooded with pure oxygen or compressed air, either being pressurized at, for example, 2 atm or higher. Both the pressure and the percentage of oxygen within the hyperbaric chamber may vary over the treatment period. For example, the patient may breathe hyperbaric oxygen or air from within the hyperbaric chamber for 60 minutes at 2 atm once a day, 20 minutes at 3 atm three times a day, or any cycle or variation in pressure thereof for a period of about five to ten days. In this embodiment, during HBO treatment no stem cell proliferation agent is administered. However, it is contemplated that stem cell proliferation agent treatment may be administered before, during, or after HBO treatment in any dose, cycle, or formulation.

In lieu of or in addition to HBO therapy, any vasodilator may be administered to the patient before, during, or after treatment with a stem cell proliferation agent, such that the concentration of nitric oxide synthase increases in the endothelial and blood cells. For example, any composition including a pharmaceutical, for example, Viagra™, or supplement, for example, Arginine, may be administered to the patient to promote vasodilation. Optionally, other medicines such as insulin or other therapies may be administered in combination with any of the above treatments before, during, or after treatment with a stem cell proliferation agent.

Following HBO treatment, or concurrently, the patient may receive treatments of stem cell proliferation agent, for example, G-CSF, or derivatives or isomers thereof, as described above. The stem cell proliferation agent may be administered in any dose, cycle, or formulation for a period of about seven days. In this embodiment, the stem cells mobilize in response to the administered G-CSF and passively migrate into the blood stream. Optionally, the proliferated stem cells may be extracted and infused into the blood stream or any location within the body. In this embodiment, HBO treatment for five days followed by treatment with G-CSF for seven days followed by HBO for five days is defined as one cycle. However, a cycle may be defined as any number of days of alternating treatments of HBO with G-CSF. In this embodiment, one cycle of treatment is administered. As shown in FIG. 3, the results of a one cycle treatment show a marked decrease in blood glucoses levels after one cycle of treatment.

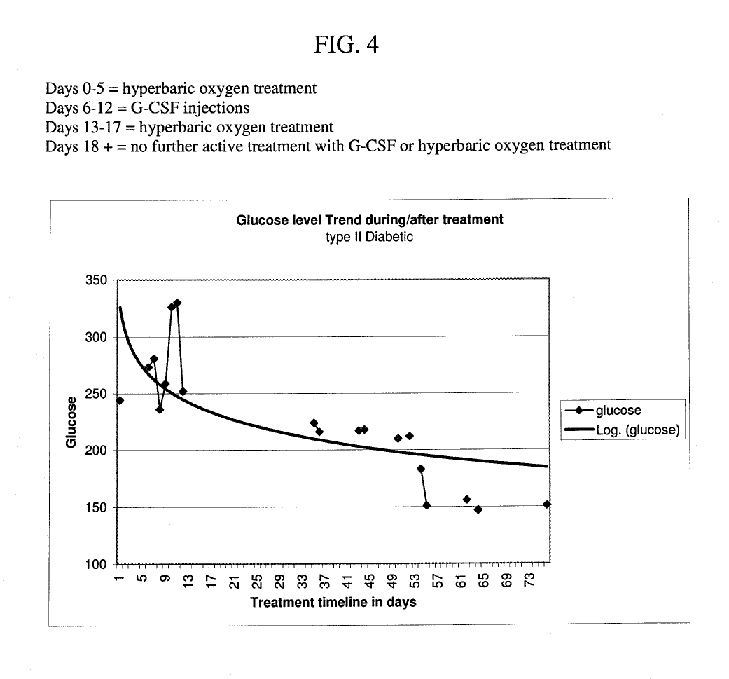

Referring now to FIG. 4, where another method of treating diabetes is shown. The method includes treating a patient with Type II diabetes with HBO treatment for a period of, for example, about five days, followed by treatments of a stem cell proliferation agent in accordance with the principles discussed above with respect to the methods in FIG. 2 and FIG. 3. In an exemplary embodiment, a patient with Type II diabetes or any metabolic or cardiovascular disorder, is either fully or partially disposed within a hyperbaric chamber such that the patient can breathe in high pressure oxygen or air in accordance with the method disclosed in FIG. 3. In this example, HBO treatment is administered for five days, followed by or concurrently with seven days of treatment with a stem cell proliferation agent, for example, G-CSF, or derivatives or isomers thereof, as described above, followed by five days of treatment. This 17 day treatment period is characterized in this embodiment as one cycle. It is contemplated that any number of treatment cycles may be performed. As shown in FIG. 4, the results of a one cycle treatment show a marked decrease in blood glucoses levels after one cycle of treatment.

Referring back now to FIG. 1, in any of the above methods the treatment markers may all be measured before, during, and after treatment to evaluate the efficacy of the treatment. For example, after each cycle of treatment, each day of treatment, or some future date, in any of the above embodiments the treatment markers may be measured (Step 108). The measured treatment markers are then compared to the target levels for the treatments (Step 110), for example the number of endothelial stem cells. If the treatment markers are below or above the predetermined target level for each, the next cycle of treatment, subsequent administration of stem cell proliferation agent, and/or duration, quality, and pressure of HBO treatment may be continued with the same target levels in order to achieve the desired target level for the treatment markers (Step 112). If not, the treatments may be terminated or the target levels may be changed (Step 114) and treatments may continue. If the target levels are achieved, treatments may continue under the same conditions or terminated (Step 116). If the treatment is not terminated, target levels may be modified depending on the desired results and treatments may continue (Step 118). For example, if the predetermined target level is achieved, it can be reset to a new target level and the treatments can resume in order to achieve the new target levels.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A method of treating endothelial cells comprising:
inducing vasodilation in a patient; and
administering a composition including a stem cell proliferation agent to the patient.

2. The method of claim 1, wherein inducing vasodilation includes administering hyperbaric oxygen to the patient.

3. The method of claim 1, further comprising extracting stem cells from the patient after administering the composition.

4. The method of claim 3, further comprising infusing the extracted stem cells into the patient.

5. The method of claim 4, wherein the extracted stem cells are returned directly into the patient's pancreas.

6. The method of claim 1, wherein inducing vasodilation includes administering a composition including a vasodilation including compound.

7. The method of claim 2, wherein the composition is administered for about seven consecutive days.

8. The method of claim 7, wherein hyperbaric oxygen is administered for about five consecutive days before administering the composition.

9. The method of claim 1, wherein the composition is included on an implant and wherein the implant is inserted into the patient.

10. The method of claim 1, wherein the composition is administered at a dose of about 300 μg to about 960 μg per day.

11. The method of claim 1, wherein the stem cell proliferation agent is G-CSF.

12. A method of treating endothelial cells comprising:
administering hyperbaric oxygen to a patient for between five and ten consecutive days;
administering a composition having G-CSF to the patient for about seven consecutive days after administering the hyperbaric oxygen;
extracting stem cells from the patient after administering the hyperbaric oxygen and administering the composition; and
infusing the extracted stem cells into the patient.

13. The method of claim 12, wherein the wherein the hyperbaric oxygen is inhaled by the patient.

14. The method of claim 12, wherein the composition is administered directly into the patient's pancreas.

15. The method of claim 12, wherein the composition is administered directly into the patient's bone marrow.

16. The method of claim 12, further comprising administering a composition including a vasodilator.

17. The method of claim 12, wherein the stem cells are infused into the patient over a period of about 24 to 96 hours.

18. The method of claim 12, further comprising readministering hyperbaric oxygen for about five to ten consecutive days after infusing the stem cells into the patient's blood.

19. The method of claim 12, wherein the composition is administered at a dose of about 300 μg to about 960 μg per day.

20. A method of treating diabetes and other metabolic disorders comprising:
measuring a blood glucose level of a patient with diabetes;
determining one or more target levels for the patient's blood glucose level;
administering hyperbaric oxygen to the patient for between five and ten consecutive days, the hyperbaric oxygen being inhaled by the patient;
administering a composition having G-CSF at a dose of about 300 μg to about 960 μg per day to the patient for about seven consecutive days after administering the hyperbaric oxygen;
extracting stem cells from the patient after the seven consecutive days of administering the composition;
infusing the extracted stem cells into the patient;
measuring the blood glucose levels;
comparing the measured blood glucose levels to the one or more target blood glucose levels; and
modifying at least one of the administering of the hyperbaric oxygen and the composition based on the comparison.
